SEQUENCE LISTING (9 sequences)

SEQ ID NO: 1 - DNA, 2179 bp, Mus sp.
tgaaactattaaattccttgctcagatttcaggaagtaaagtgtgctgttcatctcaatctctcctgtctaacccctcccctcccgatttccgggggatcaatgatagtagagagctttggggcctctggaaatcctgtggggccctgtcacttttggtccttgtatggagtcctgctaggtgtccactggagtgtgttacatctcgggacctttagaggaattcggagtgcggggctgtggctgctgtctccccattcagaagccacttgctagtagctactgaaaggctcttcattgtctcttctgctccaggaacaccggtctaggaagcagaagatgccatactccaacctgcatccagccatcccacggcccagaggtcaccgctccaaatatgtagccctcatctttctggtggccagcctgatgatcctttgggtggcaaaggatccaccaaatcacactctgaagtacctagcacttcacctagcctcgcacgaacttggactactgttgaaaaacctctgctgtctggctgaagagctgtgccatgtccagtccagttggatgtttggccttctggtcctctataagtccctaagcatgctcctgggccttcagagcttgactccagcggaagtctctgcagtctgtgaagaaaagaagttaaatgttgcccacgggctggcctggtcatactacattgggtacttgcggttgatcttaccagggctccaggcccggatccgaatgttcaatcagctacataacaacatgctcagtggtgcagggagccgaagactgtacatcctctttccattggactgtggggtgcctgacaacctgagtgtagttgaccccaacattcgattccgagatatgctgccccagcaaaacatcgaccgtgctggcatcaagaatcgggtttattccaacagcgtctacgagattctggagaacggacagccagcaggcgtctgtatcctggagtacgccacccccttgcagaccctgtttgccatgtcacaggatgccaaagctggcttcagtcgggaggatcggcttgagcaggctaaactcttctgccggacacttgaggaaatcctggaagatgtccccgagtctcgaaataactgccgcctcattgtctaccaagaacccacagacggaaacagtttctcactgtctcaggaggtgctccggcacattcgtcaggaagaaaaggaggaggttaccatgaatgcccccatgacctcagtggcacctcctccctccgtactgtcccaagagccaagactcctcatcagtggtatggatcagcctctcccactccgcactgacctcatctgaggcatgggacagccttgtctgggctctagtgatcctttagcctcctgactgagccttccttcaatggttgggggcctcagagacttcacatctccagatgagtcccacattcctgggcaagccatttatttcacctctctgagcctcaaccaaccctactatgaaaggaggtcataatgcgttccctgcccagccaaaggattttatatatgtagaagttggtgtcaatgcctggtaaacttgagagaaaggccaagtacttcccgtggatgctgcagacattccctgctctctgttgacctgtgtggatggtaccagcagacttccaaccctccagcttctggtcacgtgtgttcaatgggagcttaagtagatggcgagagggagaaggaacatttgttctgttagctgtatacaatcacagtgggctggcctgtcaactgccttcttaataaacatatctattctcagatttctagaatggcctcttccccttgtctctagcactggtatttgtgtgacactggagtactttctgtctggtctctttatatcatgtcccttgcacatggtgttggcatcaggacgtcccaaactcatgacatcacataggcgacagcatgacctgcaacctgcagaccggttgccaagacaacaggcaccatattcccaccttccacttggctcacctcccacctttacctgtgttacgtcatcttccatatcttccatacgtcttccatcttccatacgtctctctcccctgcttctctttctgctgctaccttgtctctcccttccaataaaacctcttccatgcggaactg

SEQ ID NO: 2 - DNA, 1907 bp, Homo sapiens
agaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacc

SEQ ID NO: 3 - protein, 337 aa, Mus sp.
MPYSNLHPAIPRPRGHRSKYVALIFLVASLMILWVAKDPPNHTLKYLALHLASHELGLLLKNLCCLAEELCHVQSSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 4 - protein, 260 aa, Homo sapiens
MLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 5 - DNA, 18 bp, Artificial Sequence
  Synthetic
actgcggctgcactcaga

SEQ ID NO: 6 - DNA, 22 bp, Artificial Sequence
  Synthetic
accatgccagcccatgggccac

SEQ ID NO: 7 - DNA, 18 bp, Artificial Sequence
  Synthetic
gctgtgccatgtccagtc

SEQ ID NO: 8 - DNA, 23 bp, Artificial Sequence
  Synthetic
agccagtgtccgggaggcagaag

SEQ ID NO: 9 - DNA, 18 bp, Artificial Sequence
  Synthetic
caaccgcaagtacccaat

(19) United States
(12) Patent Application Publication
(10) Pub. No.: US 2020/0291081 A1
(43) Pub. Date: Sep. 17, 2020

(54) NOVEL RECEPTORS FOR CYCLIC DINUCLEOTIDES AND METHODS AND KITS FOR SCREENING AGONISTS OR INHIBITORS THEREOF

(71) Applicant: GUANGZHOU WINKEY TECHNOLOGY CO., LTD., Guangzhou (CN)

(72) Inventors: Sujie Gu, Guangzhou (CN); Xiaobo Li, Guangzhou (CN)

(21) Appl. No.: 16/833,639

(22) Filed: Mar. 29, 2020

Related U.S. Application Data
(63) Continuation of application No. PCT/CN2018/103431, filed on Aug. 31, 2018.

(30) Foreign Application Priority Data
Oct. 11, 2017 (CN) 201710949587.4

(51) Int. Cl. C07K 14/47; C12N 15/85; C12N 5/10; C12Q 1/6888

(52) U.S. Cl. CPC C07K 14/47; C12N 15/85; C12N 5/10; A61K 45/06; C12Q 2531/113; C12Q 2561/113; C12Q 1/6888

(57) ABSTRACT

Disclosed herein is a recombinant vector being capable of expressing a novel receptor that senses exogenous cyclic dinucleotides (subtype M of STING) and meanwhile disclosed herein are a method and a kit for specifically identifying and detecting the expression of the said receptor. Further, provided herein are a medicament screening model and a kit for screening agonists or inhibitors targeting subtype M of the novel gene STING and the use thereof. Use of the screening model and the kit could simplify the screening procedure, enhance the screening efficiency and expand the range of the medicaments to be screened.

### CROSS-REFERENCE TO RELATED APPLICATIONS

This present application is a Continuation Application of PCT application No. PCT/CN2018/103431 filed on Aug. 31, 2018, which claims the benefit of Chinese Patent Application No. 201710949587.4 filed on Oct. 11, 2017, the contents of which are hereby incorporated by reference.

### REFERENCE TO SEQUENCE LISTING

The Sequence Listing is submitted as an ASCII formatted text filed via EFS-Web, with a file name of “Sequence_Listing.txt”, a creation date of Mar. 26, 2020, and a size of 11,884 bytes. The sequence listing filed via EFS-Web is part of the specification and is incorporated in its entirety by reference herein.

### FIELD OF THE DISCLOSURE

The present disclosure relates to bioengineering field and medicaments screening field, more particularly, to a receptor that senses an exogenous cyclic dinucleotide and a gene encoded by the said receptor, a vector for the said receptor, an identification method for the said receptor, an expression measurement for the said receptor and a stable cell line for the said receptor and use of the said receptor in screening medicaments as well as related kits.

### BACKGROUND OF THE DISCLOSURE

Cyclic dinucleotides (including c-di-AMP and c-di-GMP deviated from bacteria and cGAMP deviated from mammalian cells) are capable of activating signal pathway STING-TBK1-IRF3, so as to stimulate organisms to produce strong immune response. Recent researches demonstrate that exogenous cyclic dinucleotides are capable of notably increasing immune response of vaccines. Meanwhile, recent researches show that the exogenous cyclic dinucleotides exhibit excellent efficiency in anti-tumor immunology treatment. The exogenous cyclic dinucleotides could activate attacking effect of CD4 and CD8 cells against tumors with specificity in vivo and in vitro, thereby inhibiting growth of tumors. Therefore, the cyclic dinucleotides can be considered as a new generation of vaccine adjuvants and medicaments for treatment of tumors. In March, 2015, Noratis and Aduro made a deal with 750 million US dollars to co-develop a novel targeted medicament against STING.

The receptor of the cyclic dinucleotides is considered as subtype 1 of protein STING, which is located on intracellular endoplasmic reticulum (ER). Such receptor is encoded by Tmem173 gene. Since extracellular cyclic dinucleotides cannot directly pass through cell membranes while the endoplasmic reticulum is within cytoplasm, it does not know how the exogenous cyclic dinucleotides activate receptor subtype 1 of protein STING on the endoplasmic reticulum by passing through the structure of cell membranes. Therefore, current theories cannot be used to explain how the exogenous cyclic dinucleotides activate immune response within organisms. When screening inhibitors and agonists for the cyclic dinucleotides, it needs to treat cells with cell membrane permeability procedure or use liposomes to encapsulate medicaments to be screened for transfection, so as to allow the medicaments to be screened to contact subtype 1 of STING on the endoplasmic reticulum. As such, screening efficiency and the medicaments that can be screened are significantly reduced (e.g., the medicaments to be screened need to be encapsulated by liposomes), and meanwhile, there are limitations for the extracellular cyclic dinucleotides used as vaccine adjuvants and for anti-tumor immunology treatment.

The present disclosure provides a recombinant vector being capable of expressing a novel receptor that senses the exogenous cyclic dinucleotides (referred as a subtype M of STING) and a method and a kit for specifically identifying and detecting the expression of such receptor. Further, the present disclosure provides a medicament screening model and a kit targeting the new gene subtype M of STING and the use thereof.

### SUMMARY OF THE DISCLOSURE

In one aspect, the inventor, by analyzing whole transcriptome data, found that Tmem173 gene could encode subtypes (named by the inventor as subtype M of human STING and subtype M of mouse STING, hereinafter collectively referred to as subtype M of STING) of STING expressing on the cell surface, in addition to conventional type of STING (subtype 1 of STING) on endoplasmic reticulum. Through 5′RACE method, the inventor confirmed that subtype M of STING widely exists in human and mouse as well as tumor cell lines. In in vitro studies, it was found that these subtypes M of STING can directly sense extracellular cyclic dinucleotides, so as to be used as a receptor of extracellular cyclic dinucleotides. The extracellular cyclic dinucleotides can activate subtype M of STING, thereby activating the promotor of downstream type 1 interferon, and in turn facilitating up-regulation of the expression of type 1 interferon. Therefore, subtype M of STING, as the receptor of exogenous cyclic dinucleotides, is a critical target for development of vaccines and tumor immunology treatment.

The inventor found that subtype M of STING encoded by Tmem173 gene is located on the cell membranes and in the endoplasmic reticulum. The amino acid sequence of subtype M of mouse STING is set forth in SEQ ID NO:3. The amino acid sequence of subtype M of human STING is set forth in SEQ ID NO:4.

In another aspect, the present disclosure provides a recombinant vector for expressing subtype M of STING. For example, it may be Pcmv6-Entry containing DNA sequence as mentioned above.

In still another aspect, the present disclosure provides a method and a kit for specifically identifying and detecting the mRNA level of subtype 1 and subtype M of STING. The basic principle of the kit for detection comprises utilization of oligonucleotide primers with specificity, amplification of targeting nucleotides by RT-PCR amplification in RT-PCR reaction buffer solution containing reverse transcriptases, Taq DNA polymases, RNase inhibitors, deoxyribonucleoside triphosphates (dNTPs) with high quality and Mg2+ and the like, so as to quantitatively detect the mRNA level of subtype 1 and subtype M of STING with increased speed, high efficiency, and great specificity.

The kit as provided herein comprises a plurality of vials or tubes with caps for seal, each of which comprises RNA extracting reagents, RT-PCR amplification reacting solution, mixed enzymes, negative quality controls, positive quality controls, mRNA positive standards for subtype 1 and subtype M of STING, and packing boxes for separating and collecting the said vials or tubes. The mixed enzymes comprise Taq DNA polymases and reverse transcriptases (RT enzymes). The RT-PCR amplification reacting solution comprises the following oligonucleotide primers or the sequences with more than 85% homology with the following sequences or one or combination of the following sequences.

| | Forward | | Backward |
|---|---|---|---|
| Primiers | (5′ → 3′) | Primers | (5′ → 3′) |
| hIsoform | ACTGCGGCTGCACT | hIsoforms | AGCCAGTGTCCGGGA |
| M-F-1 | CAGA | M-F-2 | GGCAGAAG |
| | (SEQ ID | | (SEQ ID |
| | NO.: 5) | | NO.: 8) |
| hIsoforms | ACCATGCCAGCCCA | | |
| M-commonR | TGGGCCAC | | |
| | (SEQ ID | | |
| | NO.: 6) | | |
| mIsoform | GCTGTGCCATGTCC | mIsoform | CAACCGCAAGTACCC |
| M-F | AGTC | M-R | AAT |
| | (SEQ ID | | (SEQ ID |
| | NO.: 7) | | NO.: 9) |

The RT-PCR amplification reacting solution further comprises DNTPs, PCR Buffer and RNase inhibitors. The products obtained from RT-PCR amplification can be semi-quantified by electrophoresis (see FIGS. 1A and 1B) or can be accurately quantified by targeted sequencing using Ion Torrent.

The present disclosure further provides a method and a kit for specifically identifying and detecting the protein contents of subtype 1 and subtype M of STING. The basic principle of the kit for detection as provided herein is Western-blot technology, for quantitatively detecting protein expression level of subtype 1 and subtype M of STING with increased speed, high efficiency and great specificity.

The kit as provided herein comprises solutions for treating samples, pre-made gels for Western-blot, PVDF membranes for transmembrane, anti-subtype 1 and anti-subtype M of STING antibodies, secondary antibodies, protein positive standards for subtype 1 and subtype M of STING, and reagents for color development. Preferably, the samples are treated by the solutions for treating samples in the kit and the Western-blot experiments are carried out. The protein expression levels of subtype 1 and subtype M of STING can be semi-quantified by the stripes of targeting proteins (see FIGS. 2A and 2B) and comparing standards of subtype 1 and subtype M of STING.

In yet another aspect, the present disclosure provides a vector for preparing lentivirus of subtype 1 and subtype M of STING. Preferably, the vector can be used to stably express subtype 1 and subtype M of STING in vivo or in vitro.

In addition, the present disclosure further provides a cell line for stably expressing subtype M of STING. Preferably, the cell lines further comprise reporter genes, for indicating that the subtype M of STING is activated or inhibited. More preferably, the reporter genes are located downstream of type 1 interferon or NF kappB reaction element promoter. The present disclosure further provides a method and a kit for screening agonists and/or inhibitors for subtype M of STING by using the said cell lines.

The kit as provided herein comprises cell lines for stably expressing subtype M of STING. Preferably, the cell lines further comprise reporter genes, for indicating that the subtype M of STING is activated or inhibited. More preferably, the reporter genes are located downstream of type 1 interferon or NF kappB reaction element promoter. Preferably, the kit may further comprise a substrate on which the reporter genes act, and cell lysates. Preferably, the agonists and inhibitors for subtype M of STING can be effectively screened out by the method as shown in FIG. 4 using the kit as provided herein.

It can be seen that, the subtype M of STING is firstly found as a receptor that senses extracellular cyclic dinucleotides, which can be stably expressed in eukaryocytes. The extracellular cyclic dinucleotides exhibit great efficiency in tumor immunology treatment and development of vaccines, which indicates that the receptors for the extracellular cyclic dinucleotides, i.e., the subtype M of STING as described herein, is a critical target for tumor immunology treatment. Development and optimization of medicaments screening based on the receptor for the extracellular cyclic dinucleotides (subtype M of STING) play a key role in development of vaccines and tumor immunology treatment. Moreover, the expression level of the said receptor will be a key marker for drug sensitivity for treatment using the extracellular cyclic dinucleotides and other agonists for STING. The specific identification and the method and the kit for detection of mRNA level and protein level of subtype M of STING as described herein will play a critical role in detecting such marker. Development of vaccines and tumor immunology treatment based on the subtype M of STING can be facilitated by high-throughput screening for small molecule medicaments and screening for antibody medicaments according to the present disclosure.

So far, there are only several naturally-existing cyclic dinucleotides as agonists for STING, while no inhibitor for STING is found. The present disclosure provides a new platform for effectively, reliably and easily screening agonists and inhibitors for STING, which is suitable for high-throughput medicament screening and has significance for finding out agonists and inhibitors of the receptors for the cyclic dinucleotides.

### BRIEF DESCRIPTION OF THE DRAWINGS

FIGS. 1A and 1B show RT-PCR electrophoresis results for specific fragments of subtype M of STING from mouse brain tissues and splenic lymphocytes as well as human Hela cells and peripheral blood mononuclear cells (PBMCs), in which, Lane 1 is 100 bp Marker, Lanes 2 and 3 in FIG. 1A are the RT-PCR electrophoresis results for subtype 1 and subtype M of mouse STING from mouse brain tissues and splenic lymphocytes. Sanger sequencing results demonstrate that the stripes as shown are subtype 1 and subtype M of STING respectively. Lanes 2 and 3 in FIG. 1B are the RT-PCR electrophoresis results for subtype 1 and subtype M of human STING from human Hela cells and PBMCs. Sanger sequencing results demonstrate that the stripes as shown are subtype 1 and subtype M of STING respectively.

FIGS. 2A and 2B show the results of Western-blot using anti-STING antibodies on mouse splenic lymphocytes of two mice and human PBMC. The stripes as shown are subtype 1 and subtype M of STING respectively.

FIGS. 3A and 3B show that it can be seen that the subtype M of STING is the receptor that senses extracellular cyclic dinucleotides and activates generation of type 1 interferon by transfecting reporter plasmid of type 1 interferon into the cell lines for stably expressing subtype 1 and subtype M of STING. Both the subtype 1 and subtype M of STING can sense intracellular cyclic dinucleotides and activate generation of type 1 interferon.

FIG. 4 shows the method for high-throughput screening medicaments according to the present disclosure and comparison of such method with the conventional method.

### DETAILED DESCRIPTION OF THE DISCLOSURE

Several aspects of the disclosure are described below in details by reference to appended drawings and specific embodiments. The skilled in the art should understand that the embodiments are set forth to provide an illustration, rather than limit the scope of the present disclosure. The scope of the present disclosure is limited by the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

### EXAMPLE 1

### Construction and Identification of Gene Expression Plasmid of Subtype M of Human and Subtype M of Mouse STING and Identification of Gene Expression Level of Subtype M of Human and Mouse STING

Based on high-throughput RNA sequencing, we found that Tmem173 could encode other subtypes of STING (named as subtype M of STING), in addition to subtype 1 of STING. In order to confirm existence of subtype M of human and subtype M of mouse STINGs, we designed specific primers to specifically amplify subtypes M of human and mouse STINGs. In brief, total RNA was prepared by using RNA extraction reagents from wild-type mouse splenic cells or human PBMCs. Then the RNA was reversely transcribed to cDNA by using a reverse transcription kit (Invitrogen, 18080-051) according to the manufacturer's instructions. The primers for amplifying subtype M gene of human and subtype M of mouse STINGs were listed as below.

| | Forward | | Backward |
|---|---|---|---|
| Primer | (5′ → 3′) | Primer | (5′ → 3′) |
| hIsoform | ACTGCGGCTGCACT | hIsoforms | AGCCAGTGTCCGGGA |
| M-F-1 | CAGA | M-F-2 | GGCAGAAG |
| | (SEQ ID | | (SEQ ID |
| | NO.: 5) | | NO.: 8) |
| hIsoforms | ACCATGCCAGCCCA | | |
| M-commonR | TGGGCCAC | | |
| | (SEQ ID | | |
| | NO.: 6) | | |
| mIsoform | GCTGTGCCATGTCC | mIsoform | CAACCGCAAGTACCC |
| M-F | AGTC | M-R | AAT |
| | (SEQ ID | | (SEQ ID |
| | NO.: 7) | | NO.: 9) |

For subtype M of mouse STING, only primer mIsoform M-F/R was used for performing one-round PCR amplification in mouse splenic cells. For subtype M of human STING, nested PCR was carried out to amplify specific sequences. Human PBMCs cDNA was used as a first-round PCR template with outer primers (hIsoform M-F-1 and hIsoforms M-commonR). The products from the first-round PCR was used as the templates for carrying out the second-round PCR (using primers hIsoforms M-F-2 and hIsoforms M-commonR). The resultant sequences specific for subtype M of human and subtype M of mouse STINGs were consistent with subtypes M of human and mouse STINGs obtained by whole transcriptome sequencing. Therefore, we confirmed that subtypes M of human and mouse STINGs exist in human and mouse primary immune cells.

In order to construct recombinant expression vectors for subtypes M of human and mouse STINGs, based on the difference between subtype 1 of STING and subtype M of STING, we constructed subtype M of human STING and subtype M of mouse STING from subtype 1 of STING by performing site-directed mutagenesis in pCMV6 expression vector for expressing subtype 1 of STING. Q5 site-directed mutagenesis kit (New England Biolabs, product E0554S) was used for performing site-directed mutagenesis according to the manufacturer's instructions. The primers with specific mutation sites were designed for each mutated vector. PCR procedure for performing mutagenesis included: 94° C. for 5 min, following 37 cycles: 94° C. for 1 min, 55° C. for 30 s and 72° C. for 3 min, finally 72° C. extended for 2 min. PCR products were subject to agarose gel electrophoresis. The targeting stripes were recovered by QuickClean II gel recovery kit (Jinsirui Ltd., Product L00418) and connected by kinase-Ligase-DPNI (KLD) enzyme mixture for 10 min, and then were converted to DH5α competent cells. After 16 hours, bacterial monoclones were amplified and then the expression plasmids were purified and sequenced to confirm the accuracy of targeting sequences.

In order to construct lentivirus expression vectors for subtypes M of human and mouse STINGs, PCR amplification (94° C. for 5 min, following 40 cycles: 94° C. for 1 min, 55° C. for 1 min and 72° C. for 3 min, finally 72° C. extended for 10 min) was performed on the expression vectors for expressing subtype M of human STING and subtype M of mouse STING as mentioned above by using forward primers containing Sgf I restriction enzyme cleavage sites and backward primers containing MluI restriction enzyme cleavage sites. The targeting stripes were recovered by using QuickClean II gel recovery kit (Genscript Ltd., Product L00418) and the targeting fragments were inserted into pLenti vectors (Origene, Product RC208418L2V) with SgfI/MluI enzyme cleavage sites by using T4 DNA ligase. Then the lentivirus vectors were purified and sequenced.

### EXAMPLE 2

### Construction of Stably Transfected Cell Lines (Take HEK293T Cell as an Example

The lentivirus expression vector containing subtype M of human STING and subtype M of mouse STING as mentioned above was packed with Lenti-vpak packaging box (Origene, Product TR30022) so as to produce lentivirus particles.

HEK293T cells were incubated in Dulbecco's Modified Eagle Medium (DMEM) comprising 10% (v/v) fetal bovine serum and 10 units/ml penicillin-streptomycin solution at 37° C. The supernatants with lentivirus particles were added to the HEK293T culture solution to perform centrifugation infection. After transfection for 48 hours, the centrifuged HEK293T cells were re-suspended in complete growth medium containing 300 μg/ml G418. After two weeks, drug resistance strains occurred. GFP+ cells were sorted in FACSAria II cell sorter (BD biology scientific). The monoclonal cells were selected out by using limiting dilution method from the resultant GFP+ cells.

After obtaining cell lines for stably expressing subtype M of human and subtype M of mouse STINGs, HEK293T cell lines for stably expressing recombinant proteins were inoculated in 24-well culture plate (0.2×10⁶ cells/well) overnight. Then, the reporter genes of type 1 interferon were transiently transfected. Specifically, 3 μg reporter gene plasmids with promoter genes of type 1 interferon were diluted in 150 μl serum-free DMEM medium and mixed with 150 μl serum-free DMEM medium containing 9 μl TurboFectin Transfection Reagent (OriGene Ltd., USA). Incubate for 20 min at room temperature. The supernatants of the stable cell lines were removed and the stable cell lines were added into the combined DNA-liposome complex at 300 μl well. The 24-well plate was placed in carbon dioxide incubator and incubated at 37° C. The medium was replaced at 6 hours after transfection. The serum-free medium was replaced with complete growth medium. After 24 hours, the supernatants were removed. One group is added into complete medium containing c-di-AMP (final concentration: 30 μM, (InvivoGen, Product vac-cda)). Another group is merely added into complete medium. At 16 hour after stimulating the ligands, the activity of luciferase was determined by using the substrate of the luciferase. It was found that the group with c-di-AMP had significantly increased luciferase activity compared with control group in which only transfection was performed, which indicated that subtype M of human and subtype M of mouse STINGs could be successfully expressed and play their roles.

### EXAMPLE 3

### Method and Kit for Specifically Identifying and Detecting Subtypes M of Human and Mouse STINGs

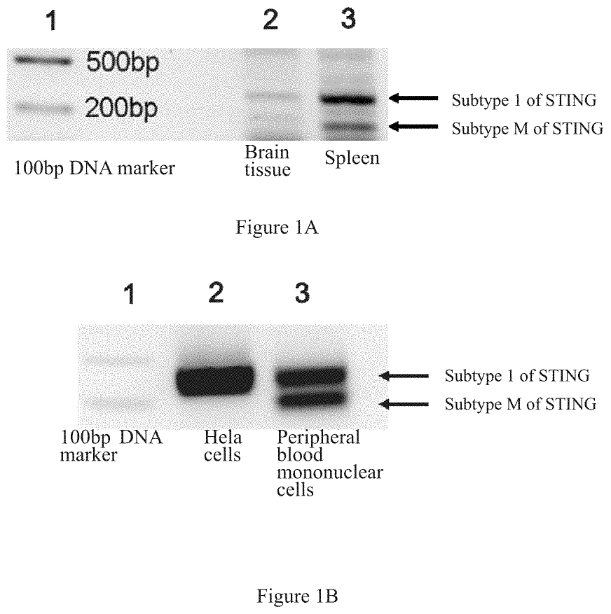

The method for detecting and identifying subtypes M of human and mouse STINGs at mRNA level comprises: extracting targeting tissues or fine RNAs with the kit as mentioned above, reversely transcribing RNA to cDNA by using RT enzyme in the kit. For the subtype M of mouse STING, one-round PCR amplification was performed merely by using primer mIsoform M-F/R. For subtype M of human STING, nested PCR was performed to amplify specific sequences. Human cDNA was used as the first-round PCR template with outer primer (hIsoform M-F-1 and hIsoforms M-commonR). The products from the first-round PCR were used as a template for performing the second-round PCR (using primer hIsoforms M-F-2 and hIsoforms M-commonR). From the agarose gel electrophoresis, the stripes of subtype 1 and subtype M of mouse STING (identified by sequencing) could be seen (FIGS. 1A and 1B). Relative quantitation can be performed by comparison of the brightness of the stripes with the brightness of the housekeeping genes. The same primers could be used for next-generation sequencing, such as Ion Torrent or Illumina platform sequencing. Accurate quantitation can be performed according to the volume of reads.

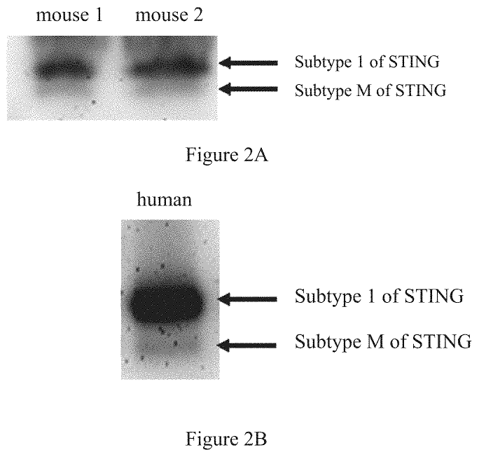

Subtype M of human and subtype M of mouse STINGs were detected and identified by conventional Western-blot method at protein level. Briefly, for preparation of targeting tissues or cells, the protein extracting solution from the kit was used to prepare protein extract (high-speed homogenizer, mortar and grinding homogenizer and ultrasonic crushing method were used). After boiling for 5 min, SDS-PAGE was performed and the proteins were transferred to nitrocellulose membranes (Bio-Rad, Product 162-0115) by using semidry process. The nitrocellulose membranes were blocked by 1×TBST containing 5% (w/v) BSA at room temperature for 2 hours. Then the nitrocellulose membranes were transferred to 1 ×TBST containing primary antibody (Cell Signaling, Product 13647, 1:1000 dilution) and 5% (w/v) BSA and wiggly incubated at 4° C. overnight, followed by washing with 1×TBST containing 5% (w/v) BSA for 3 times, each for 5 min. Then, the membranes were incubated at room temperature together with 1×TBST containing secondary antibody (anti-rabbit-HRP or anti-rabbit-AP antibody, 1:5000 dilution) and 5% (w/v) BSA for 2 hours, then washed with 1×TBST containing 5% (w/v) BSA for 3 times, each for 5 min, followed by color development and photograph. As shown in FIGS. 2A and 2B, semi-quantitative calculation for protein expression level of subtype 1 and subtype M of STING was performed according to the stripes of targeting proteins.

### EXAMPLE 4

### Subtype M of Human and Subtype M of Mouse STINGs were the Receptors that Sense Extracellular Cyclic Dinucleotides and Activate Generation of Type 1 Interferon.

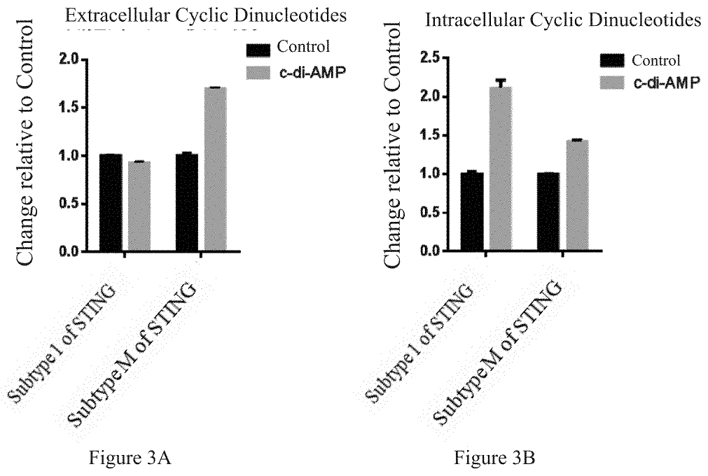

The cell lines for stably expressing each subtype of STING were constructed by transducing subtypes 1 and 3 of mouse STING and subtypes 1 and M of human STING into different cell lines lack of STING with lentivirus. Meanwhile, these cell lines stably expressed reporter luciferase mediated by the promoter of type 1 interferon. The content of type 1 interferon within the cells or supernatants could be directly detected after adding these cell lines to the medium or the medium with 30 μM extracellular cyclic dinucleotides c-di-AMP or transfecting c-di-AMP into cells by using lipofectamine 2000 for 16 hours, or cells were lysed and mixed with the substrate of the luciferase. The fluorescence intensities were read out. The ratio of the fluorescence intensities from the same cells with or without c-di-AMP was used to reflect increase of the fluorescence intensities obtained after adding c-di-AMP. It was demonstrated that subtype M of mouse STING and subtype M of human STING were the receptors that sense extracellular cyclic dinucleotides, while subtypes 1 and M of mouse and subtypes 1 and M of human STINGs could sense intracellular c-di-AMP (FIGS. 3A and 3B).

### EXAMPLE 5

### Use of Stable Cell Strains in Screening Agonists for Subtypes M of Human and Mouse STINGs

Step 1. The reporter cell lines for stably expressing subtype M of STING and the reporter genes of type 1 interferon provided by the kit mentioned above were laid on a 96-well plate, to which the medicaments to be screened were added.

For the reporter cell lines attached on the wall (e.g., HEK293T cell lines), it needed to pretreat the said cell lines with trypsin/EDTA for 10 min at 37° C. and the trypsin was neutralized in RPMI1640 with 10% fetal bovine serum. For the suspended reporter cell lines (e.g., THP-1 cell lines), centrifugation can be directly performed. After performing centrifugation for 5 min at 500 g, the cells were re-suspended in DMEM complete culture medium containing 10% fetal bovine serum, 100 U/mL penicillin and 100 μm/mL streptomycin at a cell density of 1×10⁵/ml. The cells were laid on a 96-well plate (200 μL/well) and incubated in 5% CO₂ incubator at 37° C. for use. After 16 hours, the supernatants were withdrawn and 200 μl DMEM complete culture medium containing medicaments to be screened was added, and meanwhile, a positive control group (30 μM c-di-AMP, InvivoGen, Product vac-cda) and a negative control group (merely with the culture medium without any medicaments) were established.

Since the promoter of type 1 interferon was further activated upon activating subtype M of STING, fluorescein reporter genes were added to downstream of the promoter of type 1 interferon. The expression of the luciferase reporter genes will be increased by the agonists for subtype M of STING, and the expression of type 1 interferon was also activated. The expression level of the luciferase could be measured through directly measuring the content of type 1 interferon in cells or supernatants, or through adding the substrate of the luciferase and reading the fluorescence volume generated by the substrate, thereby reflecting the activation degree for the promoter of type 1 interferon by the medicaments.

Step 2. The expression of the reporter genes upon adding the medicaments to be screened was monitored in real-time by using photometer.

At 16 hour after adding the medicaments, the supernatants were removed. 100 μl cell lysate was added to each well, followed by vibrating for 20 min. 20 μl cell lysate was mixed with 80 μl substrate of the luciferase. The fluorescence intensity of the substrate of the luciferase was measured by using photometer.

Step 3. The compounds for increasing the expression of the luciferase could be accurately and effectively screened out by using such system and the screened medicaments were agonists for subtype M of STING.

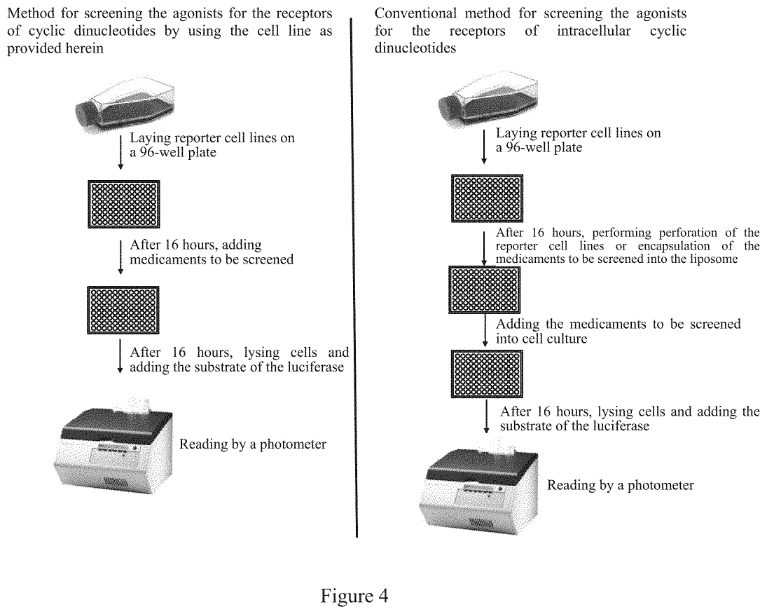

As shown in FIG. 4, as compared with the conventional method relating to the receptor STING of intracellular cyclic dinucleotides, in the method as provided herein, the stepof perforation of the reporter cell lines or encapsulation of the medicaments into the liposome was omitted, thus the procedure for screening medicaments was simplified and the range of the medicaments that can be screened was expanded (for example, there is no need to encapsulate the medicaments into the liposomes).

### EXAMPLE 6

### Use of the Stable Cell Lines in Screening the Inhibitors for Subtype M of STING

Disclosed herein is a method for high-throughput screening the inhibitors for subtype M of STING based on the reporter cell lines established as described herein. The present invention is based on the fact that identification of exogenous ligands by subtype M of human and subtype M of mouse STING can be blocked by the extracellular anti-STING protein C terminal antibody (Abcam, Product ab189430).

Step 1. The stable cell lines of subtypes M of human and mouse STINGs provided by the kit mentioned above were laid on a 96-well plate.

For the reporter cell lines attached on the wall (e.g., HEK293T cell lines), it needed to pretreat the said cell lines with trypsin/EDTA for 10 min at 37° C. and the trypsin was neutralized in RPM11640 with 10% fetal bovine serum. For the suspended reporter cell lines (e.g., THP-1 cell lines), centrifugation can be directly performed. After performing centrifugation for 5 min at 500 g, the cells were re-suspended in DMEM complete culture medium containing 10% fetal bovine serum, 100 U/mL penicillin and 100 μm/mL streptomycin at a cell density of 1×10⁵/ml. The cells were laid on a 96-well plate (200 μL/well) and incubated in 5% CO₂ incubator at 37° C. for use.

After 16 hours, the supernatants were withdrawn and 100 μl DMEM complete culture medium containing medicaments to be screened was added, and meanwhile, a positive control group (50 m/ml anti-STING protein C terminal antibody (Abcam, Product ab189430)) and a negative control group (merely with the culture medium without any medicaments or antibodies) were established. After 2 hours, c-di-AMP (final concentration, 30 μM, InvivoGen, Product vac-cda) was added.

Step 2. The expression of the reporter genes upon adding the medicaments to be screened was monitored in real-time by using photometer.

Since the promoter of type 1 interferon was further activated upon activating subtype M of STING, luciferase reporter genes were added to downstream of the promoter of type 1 interferon. The expression of the luciferase reporter genes will be increased due to the ligand c-di-AMP of subtype M of STING, and the expression of type 1 interferon was also increased. If the added medicaments could inhibit activation of the promoter of type 1 interferon and the expression of type 1 interferon per se, it could be indicated that the medicaments could inhibit the functions of subtypes M of human and mouse STINGs.

At 16 hour after adding c-di-AMP, the content of type 1 interferon in supernatants or the content of type 1 interferon within cells could be directly detected by ELISA, or the supernatants were removed. 100 μl cell lysate was added to each well, followed by vibrating for 20 min. 20 μl cell lysate was mixed with 80 μl substrate of the luciferase. The fluorescence intensity of the substrate of the luciferase was measured by using photometer.

Step 3. The compounds for inhibiting the expression of the luciferase could be accurately and effectively screened out by using such system and the screened medicaments were inhibitors for subtype M of STING.

As shown in FIG. 4, as compared with the conventional method relating to the receptor STING of intracellular cyclic dinucleotides, in the method as provided herein, the step of perforation of the reporter cell lines or encapsulation of the medicaments into the liposome was omitted, thus the procedure for screening medicaments was simplified and the range of the medicaments that can be screened was expanded (for example, there is no need to encapsulate the medicaments into the liposomes).

The foregoing is provided for illustration, and does not intend to limit the present disclosure. Any changes and modifications for the above embodiments come within the scope of the present disclosure.

1. A cell line, comprising a subtype M gene of human STING or a subtype M gene of mouse STING,
wherein:
the nucleotide sequence of the subtype M gene of human STING is set forth in SEQ ID NO:2, or is the nucleotide encoding the amino acid sequence set forth in SEQ ID NO:4,
the nucleotide sequence of the subtype M gene of mouse STING is set forth in SEQ ID NO:1, or is the nucleotide encoding the amino acid sequence set forth in SEQ ID NO:3,
the subtype M gene of human STING or the subtype M gene of mouse STING is expressed on cellular membranes.

2. The cell line of claim 1, further comprising a reporter gene for indicating that the subtype M of human STING or the subtype M gene of mouse STING is activated.

3. The cell line of claim 2, the reporter gene is located downstream of type 1 interferon or NK KappaB reaction element promoter.

4. The cell line of claim 1, comprising a recombinant expression vector whose sequence is the nucleotide set forth in SEQ ID NO:1 or SEQ ID NO:2; or comprising a recombinant expression vector whose sequence is the nucleotide encoding the amino acid sequence set forth in SEQ ID NO:3 or SEQ ID NO:4.

5. A method for screening agonists or inhibitors for the receptors of cyclic dinucleotides, comprising the following steps:
step 1. culturing the cells of claim 1;
step 2. adding medicaments to be screened, incubating the cells, detecting the content of type 1 interferon or NF KappaB within the cells or cell culture medium, or dynamically monitoring the reporter fluorescein produced by the cells to which the medicaments are added by using a photometer;
step 3. screening out the medicaments for increasing or inhibiting the expression of type 1 interferon or reporter genes.

6. The method of claim 5, wherein the medicaments to be screened are selected from the group consisting of synthetic chemical compounds, natural compounds, biological medicaments and Chinese medicine monomer.

7. A kit for screening agonists or inhibitors for the receptors for cyclic dinucleotides, comprising the cell line of claim 1.

8. The kit of claim 7, further comprising a substrate on which a reporter gene acts, and a cell lysate.

9. A medicament, comprising agonists or inhibitors for a subtype M of human STING or a subtype M of mouse STING.

10. The medicament of claim 9, wherein the medicament activates or inhibits organisms to generate immune response by activating or inhibiting signal pathway of subtype M-TBK1-IRF3 of STING.

11. The medicament of claim 9, wherein the medicament activates or inhibits organisms to generate immune response by activating or inhibiting signal pathway of subtype M-TBK1-IRF7 of STING.

12. The medicament of claim 10, wherein the medicament facilitates or inhibits the expression of type 1 interferon by activating or inhibiting signal pathway of subtype M-TBK1-IRF3 of STING.

13. The medicament of claim 11, wherein the medicament facilitates or inhibits the expression of type 1 interferon by activating or inhibiting signal pathway of subtype M-TBK1-IRF7 of STING.

14. The medicament of claim 9, wherein the medicament is an immune adjuvant.

15. The medicament of claim 9, wherein the medicament is an anti-tumor medicament.

16. The medicament of claim 9, wherein the medicament is an immune suppressor.

17. The medicament of claim 9, further comprising a pharmaceutically acceptable carrier.

18. A kit for detecting mRNA level of a subtype M of human STING or a subtype M of mouse STING, comprising RT-PCR amplification reaction solution, which comprises the following oligonucleotide primers or the sequences having a sequence homology above 85% with the following sequences or uses any one of the following sequences or combination thereof:

| | |
|---|---|
| ACTGCGGCTGCACTCAGA, | (SEQ ID NO: 5) |
| AGCCAGTGTCCGGGAGGCAGAAG, | (SEQ ID NO: 8) |
| ACCATGCCAGCCCATGGGCCAC, | (SEQ ID NO: 6) |
| GCTGTGCCATGTCCAGTC, | (SEQ ID NO: 7) |
| and | |
| CAACCGCAAGTACCCAAT. | (SEQ ID NO: 9) |

19. The kit of claim 18, further comprising RNA extracting reagents, mixed enzymes, a negative quality control, a positive quality control, a mRNA positive standard of subtypes 1 and subtype M of STING, wherein, the mixed enzymes comprise a Taq DNA polymase and a reverse transcriptase.

20. A method for detecting mRNA level of a subtype M of human STING or a subtype M of mouse STING, comprising detecting mRNA level of a subtype M of human STING or a subtype M of mouse STING by using the kit of claim 18.
